(19) United States
(12) Patent Application Publication
(10) Pub. No.: US 2016/0324564 A1
(43) Pub. Date: Nov. 10, 2016

(54) DEVICES AND TECHNIQUES FOR ABLATIVE TREATMENT

(71) Applicant: EMPIRE TECHNOLOGY DEVELOPMENT LLC, Wilmington, DE (US)

(72) Inventors: Derek Gerlach, San Francisco, CA (US); Leslie A. Field, Portola Valley, CA (US)

(73) Assignee: EMPIRE TECHNOLOGY DEVELOPMENT LLC, Wilmington, DE (US)

(21) Appl. No.: 15/107,607

(22) PCT Filed: Dec. 27, 2013
(86) PCT No.: PCT/US13/78108

§ 371 Date: Jun. 23, 2016

(51) Int. Cl. A61B 18/04

(52) U.S. Cl. CPC A61B 18/04; A61B 2018/0016

(57) ABSTRACT

Technologies are generally described for devices for ablation of a target material. The device may be a medical device and may have a medical tool portion and an ablative device portion. The ablative device portion has at least two independently controllable firing chambers. A source of fluid is in fluid communication each firing chamber. Each of the firing chambers is configured to propel the fluid to ablate a target material according to a programmed pattern of ablative treatment for the target material. Methods of ablation and use of the disclosed device are also described.

### RELATED APPLICATIONS

This application claims priority to PCT/US2013/078108, of the same title, filed on Dec. 27, 2013, which is hereby incorporated by reference in its entirety.

### TECHNICAL FIELD

The technical field of the disclosure relates generally to medical devices, and more particularly to medical devices and techniques for ablative treatment of target material.

### BACKGROUND

Typically, the removal of tissue or unwanted material from a patient requires surgery. Surgery often requires a surgeon to make an incision and cut away the tissue with a surgical knife. A surgeon may be faced with competing requirements between inadequate removal of the unwanted material, and unnecessary removal of healthy surrounding tissue. Either over-treatment or under-treatment may result in undesirable patient outcomes.

Ablative techniques have been used in gallstone, cancer, and cardiac treatments often as a part of minimally invasive surgical techniques, with the goal of treating these diseases with less overall trauma to the patient and lower cost. Ablative techniques including powerful lasers, rotary gears and saws are some of the standard practices for reduction and removal of gallstones and cancerous tumors. These techniques can be imprecise, and can cause considerable collateral damage to the patient.

Current devices and techniques used by surgeons to treat conditions such as gallstones, cancerous tumors, and related conditions, may create potential for damage that could be minimized with a more localized technique. As a result, current devices and techniques may not always result in desired outcomes.

Unless otherwise indicated herein, the materials described in this section are not prior art to the claims in this application, and are not admitted to be prior art by inclusion in this section.

### SUMMARY

Technologies are generally described for devices for ablative treatment of a target material. The device may be a medical device and may have a medical tool portion and an ablative device portion. The ablative device portion has at least two independently controllable firing chambers. A source of fluid is in fluid communication with each firing chamber. Each of the firing chambers is configured to propel fluid from the source of fluid to ablate a target material according to a programmed pattern of ablative treatment for the target material. Methods of ablation and use of the disclosed device are also described.

The foregoing summary is illustrative only, and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an ablative device of the prior art and a target material in a patient to be ablated;

FIG. 2 is a perspective view illustrating an embodiment of a medical device having a medical tool portion and an ablative device portion for ablating a target material in a patient according to this disclosure;

FIG. 3A is a frontal perspective view illustrating an embodiment of an ablative device of the present disclosure for illustrative use as the ablative device portion of the medical device shown in FIG. 2;

FIG. 3B is a rearward perspective view of the ablative device shown in FIG. 3A;

FIG. 4A shows a target to be ablated by an ablative treatment and a virtual map overlying that target that the ablative device of this disclosure uses to pattern the ablation of the target according to this disclosure. FIGS. 4B, 4C, and 4D illustrates how the virtual map of FIG. 4A changes at three points in time according to a patterned program for delivering the ablative treatment according to this disclosure;

FIG. 5 is a perspective view of an embodiment of a firing chamber of the ablative device shown in FIGS. 3A and 3B;

FIG. 6 is a perspective view illustrating another embodiment of a medical device having a medical tool portion and an ablative device portion with firing chambers configured to fire ablative material in different directions according to this disclosure;

FIG. 7 is a perspective view illustrating another embodiment of a medical device having an ablative device portion incorporated within a medical tool portion; and

FIG. 8 is a flowchart illustrating a method of ablating a targeted material with an ablative device according to the present disclosure; in which all of the figures are arranged according to at least some embodiments presented herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and computer program products related to medical devices and techniques for ablative treatment of target material.

Technologies are generally described for devices and methods for ablative treatment of a targeted material. The device may be a medical device and may have a medical tool portion and an ablative device portion. The ablative device portion has at least two independently controllable firing chambers. A source of fluid is in fluid communication with each firing chamber. Each of the firing chambers is configured to propel fluid from the source of fluid to ablate a target material according to a programmed pattern of ablative treatment for the target material. Methods of ablation and use of the disclosed device is also described.

In describing more fully this disclosure, we make reference to the accompanying drawings, in which illustrative embodiments of the present disclosure are shown. This disclosure may, however, be embodied in a variety of different forms and should not be construed as so limited.

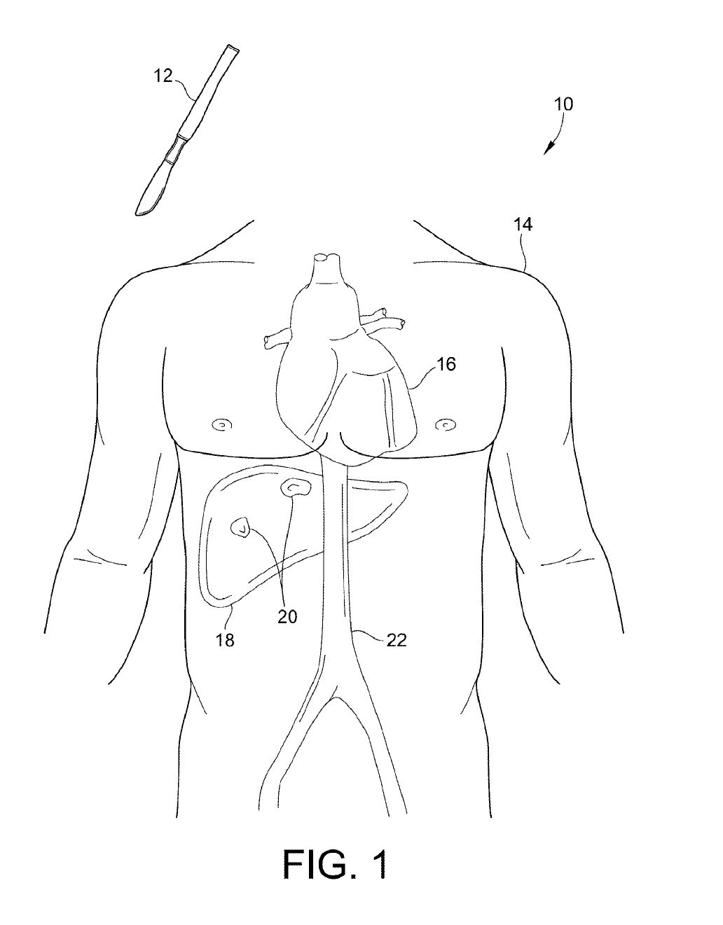

FIG. 1 shows a medical device in the form factor of a surgical knife 12 of the prior art and target materials 20, illustratively cancerous tumors, in a patient 14 to be removed. The removal of unwanted tissue from patient 14 typically requires surgery, generally depicted as 10. The surgery 10 requires a surgeon to make an incision and cut away the unwanted tissue with surgical knife 12. The surgeon in this example is faced with competing requirements between inadequate removal of the unwanted material and unnecessary removal of healthy surrounding tissue. In this example, the cancerous tumors 20 are to be removed from a liver 18.

As is well known in the art, inadequate removal of tumors 20 may result in insufficient treatment of the cancer. Any unnecessary removal of healthy liver 18 may result in unnecessary damage to liver 18. Additionally, other tissue and organs, such as heart 16 and aorta 22, are in very close proximity to cancerous tumors 20, and may be subject to damage when performing surgery 10 with surgical knife 12. Other surgical procedures, for example, treating conditions such as gallstones, cancerous tumors, and related conditions, with current medical devices and techniques used by surgeons may also not result in desired outcomes. Either over-treatment or under-treatment may result. As will be appreciated by one skilled in the art, for some types of surgery, commonly used surgical tools and techniques for ablative treatment may lead to undesirable patient outcomes.

Having thus introduced background on surgical tools and techniques, we now turn to features that are provided by this disclosure.

Technologies are generally described for devices and methods for ablating a target material. The term ablate or ablating as used herein generally means excising, removing, amputating, or otherwise altering or destroying a portion of the structure and/or function of the ablated material. An ablating of a biological material may cause its biological function to be altered or destroyed. A material that has been ablated may be removed from the body or may remain in place, such as for the body to absorb. Ablating may be carried out by a number of techniques such as by erosion, melting, evaporation, vaporization, or other physical or nonphysical techniques.

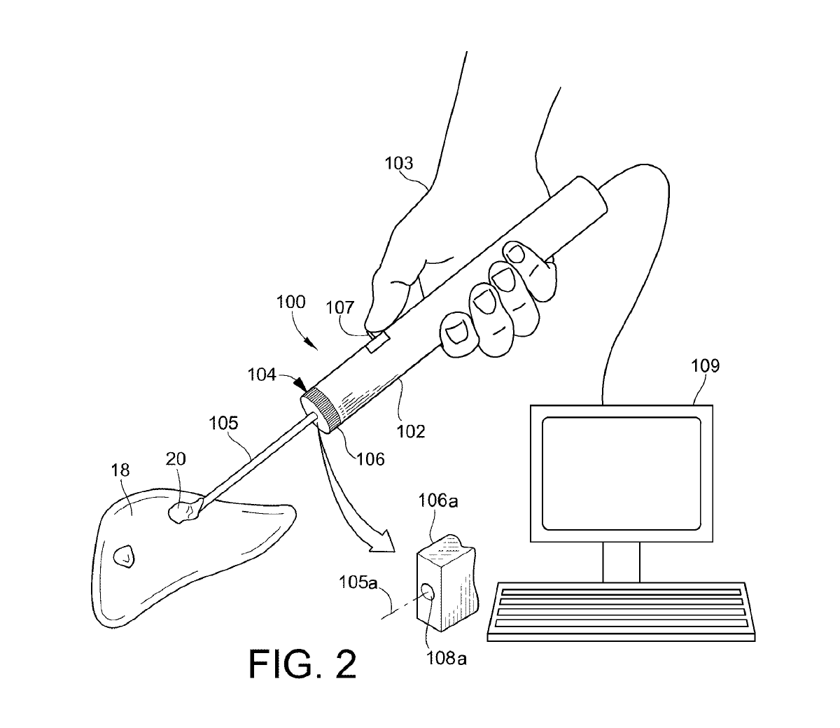

FIG. 2 shows a medical device 100 of this disclosure having a medical tool portion 102 coupled with an ablative device portion 104 (also referred to as “ablative device”). In this illustrative example, the medical tool portion is configured to be held by hand 103. The medical tool portion is provided with a trigger 107 which may be manually activated by a user.

A controller 109 is provided for controlling the medical device 100. The controller is responsive to the trigger 107.

Medical device 100 may have a variety of configurations and may be configured for a variety of procedures. For example, medical device 100 may be configured to treat a condition such as a tumor, stone, gallbladder, kidney, obstruction, cancer, cardiac, liver, gastrointestinal tract, pulmonary, or combinations thereof. Medical device 100 may be configured for a variety of procedures such as aspiration, colonoscopy, or cauterization. The medical tool portion 102 may be configured as a catheter tip, an endoscopic device, a laparoscopic device, a stent, or in other ways as will be apparent from this disclosure.

Ablative device portion 104 of medical device 100 includes a plurality of independently controllable firing chambers 106. One independently controllable firing chamber 106a of the independently controllable firing chambers 106 is shown in FIG. 2 in exploded view. Each one of the independently controllable firing chambers 106 is configured to operate like the one independently controllable firing chamber 106a and hence each one of the independently controllable firing chambers 106a is in fluid communication with a source of fluid, not shown. The one independently controllable firing chamber 106a is configured to propel fluid 105a from inside the firing chamber, through an orifice 108a, toward a target material 20, to ablate target material 20 according to a programmed pattern of ablative treatment for target material 20. The programmed pattern is programmed into the controller 109 prior to the start of an ablative treatment. User actuation of trigger 107 initiates the ablative treatment. The ablated material and fluid may be removed from the body through suction, drainage, vacuum removal, aspiration, and/or other techniques.

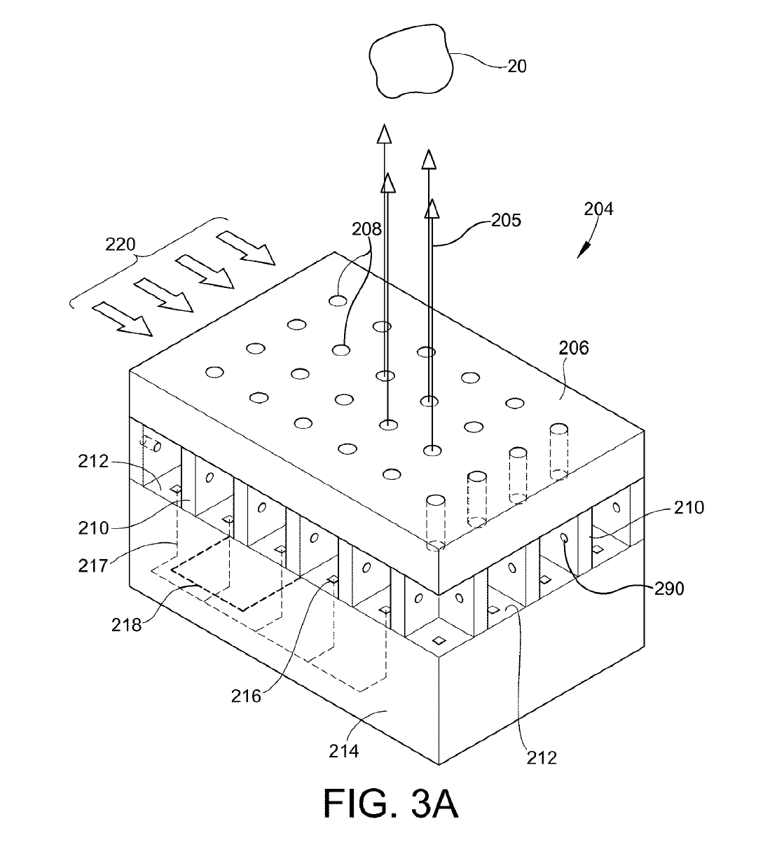

Turning now to greater details on this disclosure, FIGS. 3A and 3B show a frontal perspective view and a rearward perspective view of ablative device portion 204 of this disclosure. Ablative device portion 204 may be a component assembled with a medical tool portion to form a medical device as shown in FIG. 1, or may be integrated with a medical tool to provide a medical device. However, it is to be understood that ablative device portion 204 may be adapted or configured to be independently used as a stand-alone medical device, without a medical tool portion forming a part of the medical device.

FIG. 3A shows ablative device portion 204 in fluid communication with a fluid 220 coming into the ablative device portion 204 from a fluid source (not shown). Ablative device portion 204 includes a plurality of independently controllable firing chambers 212, each in fluid communication with the fluid source. Each firing chamber of the independently controllable firing chambers 212 is configured to propel fluid inside the firing chamber received from the fluid source through an array of orifices 208 toward a target material to ablate the target material according to a programmed pattern of ablative treatment for the target material as described below.

The fluid source that provides fluid to the ablative device portion may be a reservoir located on or remote from the medical device. For example, the reservoir may be located on the medical device. The reservoir may be configured to be a part of the ablative device portion or a part of the medical tool portion of the medical device. Alternatively, the reservoir may be located away from the medical device and connected to the ablative device portion by a tube or conduit to allow fluid to communicate from the reservoir to the ablative device portion 204.

It will also be appreciated by one skilled in the art that the fluid source may include a plurality of fluid sources and hence a plurality of fluid sources may be used to provide fluid to the independently controllable firing chambers 212 of this disclosure. For example, ablative device portion 204 may be partitioned into a first set of independently controllable firing chambers and a second set of independently controllable firing chambers. The first set of independently controllable firing chambers may be in fluid communication with a first fluid source of the plurality of fluid sources and the second set of independently controllable firing chambers may be in fluid communication with a second fluid source of the plurality of fluid sources. The number and configuration of the fluid source 220 will be apparent to one skilled in the art upon reading the present disclosure.

Illustratively, the fluid used with the ablative device portion of this disclosure is pure water. Alternatively, the fluid may be a purified water substantially void of solids. It is important that the fluid be substantially void of large molecules so as not to plug up any one or more orifices of the array of orifices 208 through which the fluid must pass. As previously explained, the fluid passes through the one or more orifices when and depending upon which one or more of the independently controllable firing chambers 212 is fired. Fluids substantially void of excessively large molecules or particles may include an aqueous solution of water and a biologically active ablation material, bactericidal material, ethanol, chemotherapy material, anti-inflammatory material, anesthetic material, osmotically balanced fluids, osmotically balanced saline solution, surfactants, or other physiologically compatible fluid.

In at least one embodiment, at least one of the independently controllable firing chambers 212 is configured to propel a biologically active ablation fluid. A biologically active ablative fluid may illustratively include an aqueous solution of water and an antiseptic or other antimicrobial substance such as alcohol, quaternary ammonium compounds, boric acid, brilliant green, chlorhexidine gluconate, hydrogen peroxide, iodine, mercurochrome, manuka honey, octenidine dihydrochloride, phenol (carbolic acid), sodium chloride, sodium hypochlorite, calcium hypochlorite, and sodium bicarbonate (NaHC03).

The fluid for use with this disclosure is selected to advantageously have a surface tension and contact angle in the proper ranges to allow flow into the plurality of independently controllable firing chambers 212; properties that minimize or eliminate gases to be entrained in the fluid; and be substantially void of large molecules to avoid plugging of the one or more orifices of the array of orifices as previously described.

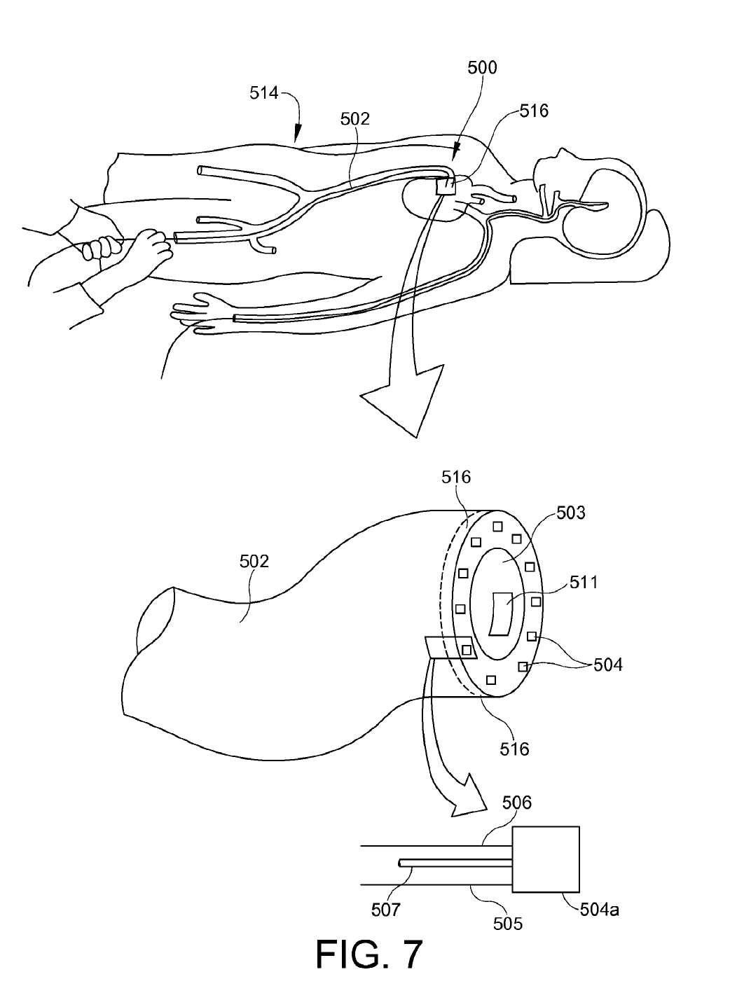

The ablated material and fluid is removed from the body through suction, drainage, vacuum removal, aspiration, and/or other techniques as described in greater detail in FIG. 7 below.

Each one of the independently controllable firing chambers 212 may be positioned substantially equidistant from adjacent ones of the independently controllable firing chambers 212. Alternatively, one or more of the independently controllable firing chambers 212 may be positioned at different distances from adjacent ones of the independently controllable firing chambers. The precise positioning of each one of the independently controllable firing chambers may depend upon the desired positioning with respect to the ablative target of the one or more of the orifices of the array of orifices through which the independently controllable firing chambers propel fluid. In one configuration, all or substantially all of the orifices of the array of orifices may be positioned to have an axial direction that lies perpendicular to a plane formed by the ablative device portion 204. In another embodiment, a first set of one or more individual orifices of the array of orifices may be positioned to have an axial direction that lies perpendicular to the plane formed by the ablative device portion while, a second set of one or more individual orifices of the array of orifices may be positioned to have an axial direction that lies at an angle other than 90 degrees to the plane formed by the ablative device portion. One skilled in the art may appreciate that a desired positioning and axial direction of each orifice of the array of orifices in the ablative device portion 204, and hence the positioning of the independently controllable firing chambers 212 in the ablative device portion 204, with respect to each other, may depend upon the intended ablative treatment.

Ablative device portion 204 may be activated or configured to propel liquid from firing chambers 212 in a variety of ways. For example, ablative device portion 204 may be heat activated. In at least one embodiment of ablative device portion 204, ablative device portion 204 comprises resistive heater elements 216 as shown in FIG. 3A.

Illustratively, each one of the resistive heating elements 216 may be a thermal activator element configured to rapidly heat a thin layer of the fluid 220 adjacent the each one of the resistive heating elements to a temperature of about 300 Celsius (C) (the temperature scale at which water freezes at 0° and boils at 100° at standard conditions). In view of this disclosure, one skilled in the art will appreciate that there are other ways in which resistive heating elements 216 may be configured.

Resistive heater elements 216 may be arranged in an array such that one resistive element of the resistive heater elements 216 is provided in each of the independently controllable firing chambers 212. Each one resistive element of the resistive elements 216 of an associated one of the independently controllable firing chambers 212 illustratively overlays a wall of the associated one of the independently controllable firing chambers 212; the wall being configured to lie opposite the associated one or more orifices of the array of orifices 208 that is provided to the independently controllable firing chambers 212 as previously described. Hence, each one of the resistive heater element 216 may be associated with one of the independently controllable firing chambers 212 and may be configured to generate heat under the influence of an applied electric current. Control of each one of the independently controllable firing chambers 212 may thus occur by selective heating of one or more of the resistive heating elements 216 in response to electrical signals applied by a controller 218, shown in FIG. 3B, according to the programmed pattern as described below.

In this illustrative embodiment, the controller is located in the ablative device portion but the controller may also be remotely located as illustrated in FIG. 2 above.

Ablative device portion 204 may comprise a substrate 214 configured to attach to a medical tool portion (not shown). The independently controllable firing chambers 212 are illustrative formed on top of substrate 214, as explained below, and the substrate 214 is configured to provide structural foundation for the firing chambers 212. Alternatively, the independently controllable firing chambers 212 may be formed inside the substrate 214 such as by using etching techniques known in the semiconductor industry or in other ways known to one skilled in the art in view of this disclosure.

In the illustrative example of FIG. 3A, the independently controllable firing chambers 212 are formed along a top surface of the substrate 214 with the top surface of the substrate 214 defining a floor or wall of the independently controllable firing chambers 212 against which resistive heater elements 216 lie (i.e., the resistive heater elements 216 lie on top of this floor or wall) as shown in FIG. 3A and previously described. Alternatively, the resistive heater elements 216 may lie inside the previously described floor wall of the independently controllable firing chambers 212, for generating predetermined heating patterns under the influence of predetermined electric current patterns applied to the resistive heating elements 216, according to a programmed pattern.

Where, as in FIG. 3A, the independently controllable firing chambers 212 are formed on top of the surface of the substrate 214, a barrier grid 210 may be provided on substrate 214 to define sidewalls of each one of the independently controllable firing chambers 212. Illustratively, barrier grid 210 may comprise a fine mesh or screen having a mesh or screen size sufficiently small enough to contain fluid within the firing chamber. Each grid of the barrier grid 210 may be provided with one or more openings 290 between adjacent ones of the independently controllable firing chambers 212 to allow for the flow of fluid 220 between the adjacent ones of the independently controllable firing chambers 212.

A top or ceiling to each one of the independently controllable firing chambers 212 may be provided by an orifice plate 206. As shown in FIG. 3A, the orifice plate 206 may overlay barrier grid 210. The orifice plate 206 is provided with the previously described array of orifices 208 which provide the one or more openings for the discharge of fluid from the independently controllable firing chambers 212.

As previously explained, orifice plate 206 includes the array of orifices 208, wherein each orifice of the array of orifices 208 is associated with one of the independently controllable firing chambers 212. Each orifice of the array of orifices 208 may be configured to allow the passing of the fluid 220 that is propelled from inside associated one of the independently controllable firing chambers 212, toward a target material according to a programmed pattern. As previously described, in at least one embodiment, each one of the independently controllable firing chambers 212 is configured to propel fluid 220 inside independently controllable firing chambers 212 outwardly from the ablative device portion in response to heating of a thin layer of the fluid 220 in independently controllable firing chambers 212 by resistive heating element 216 to a temperature of about 300° C. The outwardly propelled fluid 205 may be a single stream or a plurality of streams, depending upon how many of the independently controllable firing chambers 212 are actuated. The pattern of independently controllable firing chambers 212 actuated and hence the pattern of the outwardly propelled fluid 205 is controlled by the controller 218 as described below. As previously explained, in this illustrative embodiment, the controller is located in the ablative device portion but the controller may also be remotely located as illustrated in FIG. 2 above.

The term independently controllable firing chambers 212 as used herein refers to a bounded volume having an orifice, a fluid inlet, and a fluid activator.

FIG. 3B shows a rearward perspective view of the ablative device portion 204 shown in FIG. 3A. The rearward view shows the substrate 214 overlaying the independently controllable firing chambers 212 and the orifice plate 206. The substrate 214, independently controllable firing chambers 212, and the orifice plate 206 are configured to operate in the manner previously described. As previously described, each one of the independently controllable firing chambers 212 has a an opening 290, or fluid inlet illustratively provided by the barrier grid 210, for allowing entry of fluid 220 from a source of fluid into each one of the independently controllable firing chambers 212. Each one of the independently controllable firing chambers 212 has one or more orifices of the array of orifices 208 configured for allowing egress of fluid from the independently controllable firing chambers 212 to be propelled at high velocities toward an ablative target, due to the heating effects of the resistive heating elements 216 of this disclosure.

As FIG. 3B shows, ablative device portion 204 further includes a controller 218, illustratively mounted on a printed circuit board 223, configured to independently control the propelling of fluid from each firing chamber 212, according to a programmed pattern. In this illustrative embodiment, the controller is located in the ablative device portion but the controller may also be remotely located as illustrated in FIG. 2 above. A signal path 217 connects the controller to each resistive heating element of resistive heating elements 216 for communicating an electrical signal to each resistive heater element. Ablative device portion 204 may also have a memory unit 219, for storing the instance of instructions. The independently controllable firing chambers 212 may occur by the controller executing a set of instructions of the instance of instructions.

Controller 218 on printed circuit board 223 may reside inside of substrate 214 as shown in FIG. 3B, or be mounted on substrate 214 or may be located remotely from substrate 214. In at least one embodiment, the substrate 214 includes the controller 218. Printed circuit board 223 may illustratively be made from a flexible material and electronics. In other words, the printed circuit may be a flex circuit, which is a technology for assembling electronic circuits by mounting electronic devices on flexible plastic substrates, such as polyimide and PEEK Film. Additionally, flex circuits can be screen printed silver circuits on polyester. By making the ablative device portion 204 more flexible, it may be possible for the ablative device portion to better fit the form factor of the medical device to which is assembled. A more flexible ablative device portion may be advantageous in certain procedures such as threading of the medical device through an artery as described below.

As previously described, the ablative device portion may be formed as an integral part of a medical device. Alternatively, ablative device portion 204 may be formed as a separate device that is configured to be adapted with or attached to a medical device. In an illustrative example, a bottom surface of the substrate 214 may be attached to the medical device by sonic welding, use of an adhesive, mechanical coupling between interlocking elements on the substrate and medical device, or by other means.

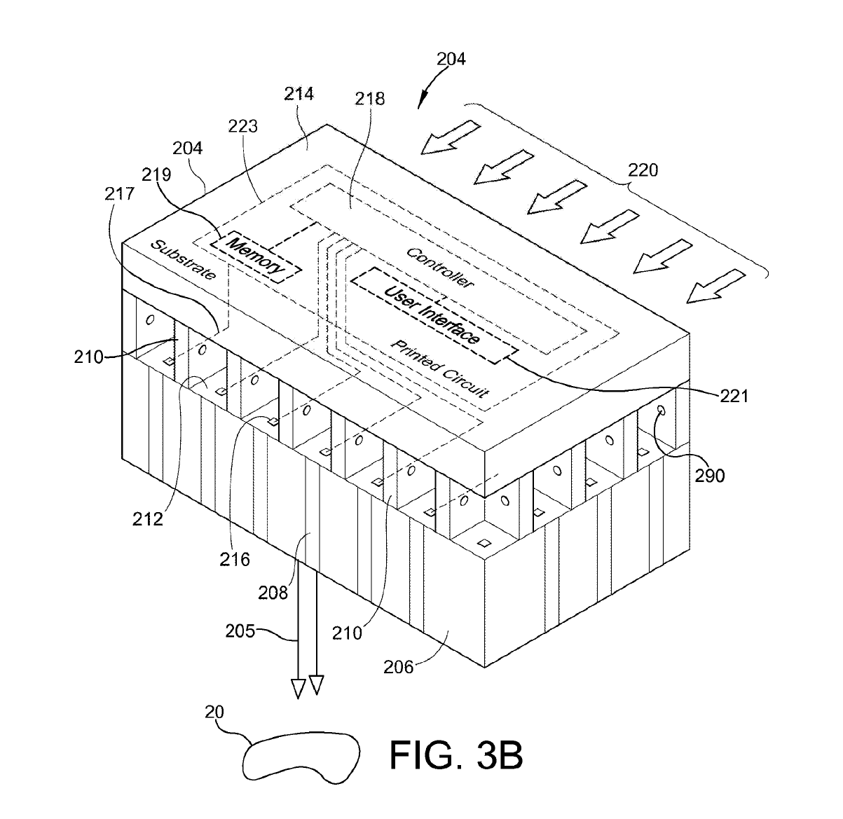

FIG. 4A shows a target such as a cancerous tumor on a liver to be ablated by an ablative treatment of this disclosure. FIG. 4A also shows a virtual memory map 260 overlying that target. The virtual memory map is a map in a memory unit of a controller (shown in FIGS. 2, 3A, and 3B) of an ablative device portion that contains the pattern of instructions to be used by the ablative device of this disclosure in performing an ablative treatment.

FIGS. 4B, 4C, and 4D illustrate how the virtual memory map of FIG. 4A changes at three points in time according to a patterned program for delivering the ablative treatment according to this disclosure. As shown in FIGS. 4B, 4C, and 4D, each memory map in FIGS. 4B, 4C, 4D, comprises an X by Y grid of logical ones and zeros. Each cell in each grid represents one firing chamber of an array of independently controllable firing chambers 260. A logical one in a cell of the grid indicates that the firing chamber associated with that cell is to be activated. A logical zero in the cell indicates that the firing chamber associated with that cell is not to be activated.

Memory map of FIG. 4B shows the pattern of ablative treatment at time t=1 as a collection of four logical ones in the grid which means that the four firing chambers of the ablative device of this disclosure that is associated with the cells containing the logical one are activated during time t=1. FIG. 4C shows the pattern of ablative treatment at time t=2 in which more firing chambers have been activated as evidenced by the larger number of cells that contain a logic one. In FIG. 4D, which shows the pattern of ablative treatment at time t=3, even more firing changers have been activated as evidence by the even larger number of cells that contain a logic one. FIGS. 4B-4D thus show an ablative treatment according to this disclosure in which at time t=1, there is a focused delivery of ablative fluids whereafter more ablative streams are activated at time t=2, and even more ablative streams are activated at time t=3. This might be a desired programmed pattern where the ablative target has a rounded shape. The ablative treatment illustrated thus provides the most ablative treatment near the center of the target since that is the portion of the target that protrudes the most from the plane of the target. At time t=2, the ablative treatment has spread to also include a portion around the center of the target which protrudes more from the plane of the target than does the peripheral portion of the target. At time t=3, the ablative treatment has spread to also treat the peripheral portion of the ablative target.

In practice, the ablative device portion would be positioned above the tumor 254 shown in FIG. 4A and the ablative treatment would be initiated. The programmed changing of patterns of the delivery of high velocity fluids during the ablative treatment according to an ablative treatment plan allows the ablative device portion of this disclosure to steer the delivery of high velocity fluids first toward a rounded protrusion of tumor 254 using the programmed pattern shown in FIG. 4B, then across a broader region about and including that protrusion according to the programmed pattern shown in FIG. 4C, and finally to an even broader region about and including the protrusion according to the programmed pattern shown in FIG. 4D. In this example, the ablative device portion may be held stationary throughout the ablative process with only the pattern of high velocity fluids propelled from the ablative device portion changing according to the preprogrammed pattern.

It will be appreciated that the ablative device portion may itself be moved or steered during the ablative treatment. For example, the instance of the ablative treatment procedure executed by the controller shown in FIG. 2 may begin with a set of preprogrammed ablative patterns such as shown in FIGS. 4B, 4C, and 4D. The instance may then instruct that the ablative device portion be moved to a new coordinate with respect to the ablative target to continue a different programmed pattern of ablative treatment. The ablative device portion may be moved to a new coordinate robotically by movement of the ablative device portion along a plane or axis of an assembly to which the ablative device portion may be affixed in this illustrative example. The ablative device portion may also be moved to a new coordinate manually such as explained in connection with FIG. 7 below.

One skilled in the art will appreciate that by changing the arrangement of patterns during different periods of time of an ablative treatment, a wide range of ablative treatments is possible by the teachings of this disclosure. It will also be appreciated that by changing the coordinates of the ablative device portion with respect to a target, the ablative device portion of this disclosure may provide further precision in the ablative treatment of a patient's tumor.

### Example

In one illustrative example of the present disclosure, an ablative device is configured using MEMS thermal inkjet-like ablation techniques using miniaturized devices that may be brought to the area to be treated as part of a localized surgical tool. This ablative device may be illustratively used in open surgery and the ablative device may be a part of a medical device such as a catheter tip or other endoscopic or laparoscopic device. More specifically, the ablative device is configured using MEMS thermal inkjet (TIJ) printing technologies wherein liquid droplets may be propelled by the use of superheat on a micro-scale in an extremely well-targeted and high resolution way. The energy for the localized ablation arises from the very controllable superheat explosion of the picoliter-scale (pL is a unit of measurement equal to one trillionth of a liter) volume of fluid (such volumes are attainable in TIJ printheads) in the thermal inkjet-like device, which may not damage surrounding tissues by overheating, overcooling, overgrinding, etc. In a localized area within the individual firing chambers, the liquid in a very thin layer is heated to approximately 300° C. over microseconds, within an extremely small volume of the fluid immediately adjacent to the resistive heater in the firing chamber, producing an energetic explosion controlled and directed over an extremely small area.

The area to be ablated may be controlled from sizes analogous to a few pixels to the sizes of hundreds, thousands, or more pixels activated in parallel to provide ablation over a larger controlled area. One illustrative ablative device is configured using the specifications of a commercially available inkjet printhead which has a 1200 dpi resolution (dpi means dots per inch), equivalent to 21 μm per dot (μm means micrometer which is one-millionth of a meter). Each firing chamber when fired propels fluid at a high velocity to target an area corresponding roughly to a 20 μm diameter dot, and if 100 contiguous firing chambers were fired, an area roughly 100 times larger could be targeted. If all the firing chambers of this device were fired at once, it would cover an area roughly equivalent to the total active area of 0.5″×0.85″ (″ means inches), which is roughly 1.3 cm×2.2 cm (cm means centimeter which is one-hundredth of a meter). This area may be large enough, and at the same time may have enough resolution, to be suitable for surgical applications.

A small resistive heater element is made using a thin film technology analogous to inkjet technology, for which 0.2 W (W means watts which is a unit of power) of extractable energy may be obtained from a 37 V pulse (V means volts which is a unit of electromotive force) applied over 6 microseconds. In the current disclosed ablative device, such energy may be used to propel droplets of liquid at the treatment site to generate an extremely localized energetic hydromechanical ablation technique. The ablative device of this example may be used at a distance from the area to be treated, which may render the device of greater utility than if contact were required. This may allow a surgeon to observe what the treatment site looks like during the treatment session.

Extractable energy from a firing chamber of the present example may generate a significant acoustic pressure wave of 200 mBar (0.2 Atm) (mBar means millibar and Atm means atmospheres which are units of pressure) at its peak, at 2 mm away (mm means millimeter which is one thousandth of a meter) from the firing chamber. In at least one aspect, in which a longer platinum wire is used to apply the superheat to the liquid thin layer surrounding the wire, an electrical energy input of 24 W for 8 microseconds may yield 0.5 W of extractable energy, and 6.2 Bar (6 Atm) of pressure. An acoustic pressure wave of 25 mBar may be produced at its peak at 20 mm (2 cm) away. Such pressure waves may be achieved even without including any particular device structure designed to intentionally direct the pressure wave or propelled fluid. A resistive heater in the firing chamber rapidly heats the fluid (which could be water, water with additives, or other liquids) over a time period of roughly microseconds so as to favor homogeneous nucleation (superheating) before any significant lower-energy heterogeneous nucleation (traditional boiling as seen in cooking) has a chance to occur. Localized pressures of about 130 atmospheres may be generated. In this embodiment, an ablative device has 36,000 nozzles or orifices and is configured to operate at about 48 kHz to provide 1200 dots per inch (dpi) with 2 pL droplets, in roughly a 0.5″×0.85″ area.

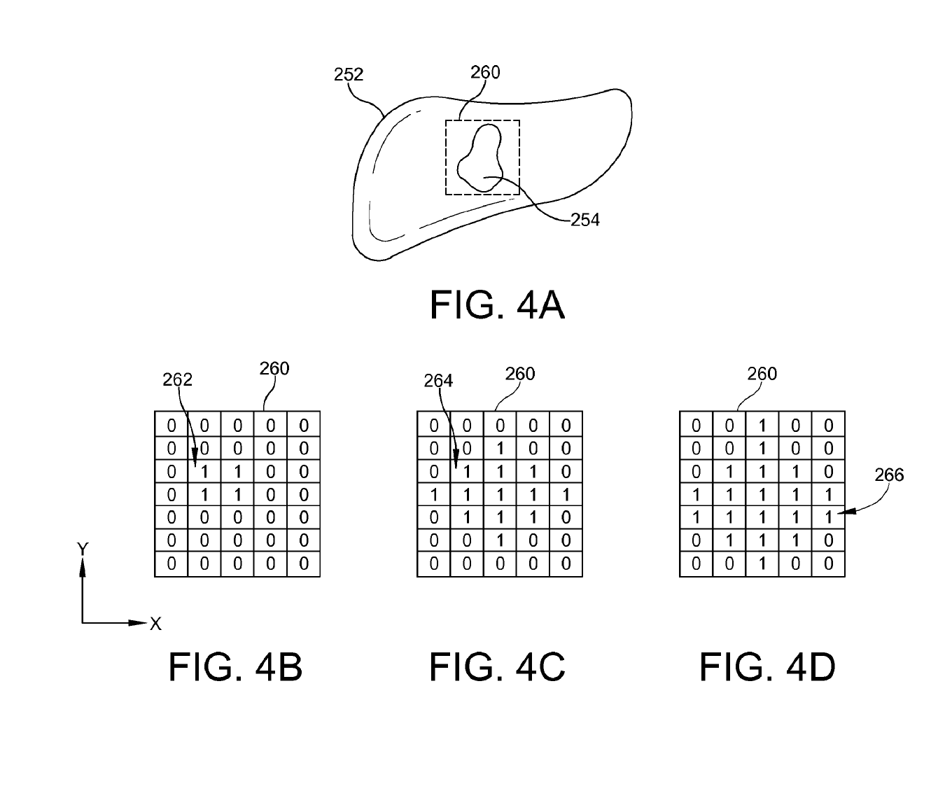

FIG. 5 shows a close-up view of a single independently controllable firing chamber 304 of an ablative tool 300 (also referred to as ablative device portion or ablative device) of the present disclosure. The independently controllable firing chamber 304 comprises a fluid inlet for allowing entry of fluid 320 from a source of fluid into independently controllable firing chamber 304. An orifice outlet 308 provided in orifice plate 306 is configured for allowing the egress of fluid from firing chamber 304. Fluid activator 316 is configured to activate the fluid in firing chamber 304 and propel the fluid 305 at a high velocity out of orifice 308 to ablate a desired material according to a programmed pattern of ablative treatment for a target material. The elements of the single independently controllable firing chamber operates in the manner described in connection with FIGS. 3A, 3B above. A plurality of independently controllable firing chambers provides an ablative device of this disclosure for use in providing manual or programmed ablative treatments to a target.

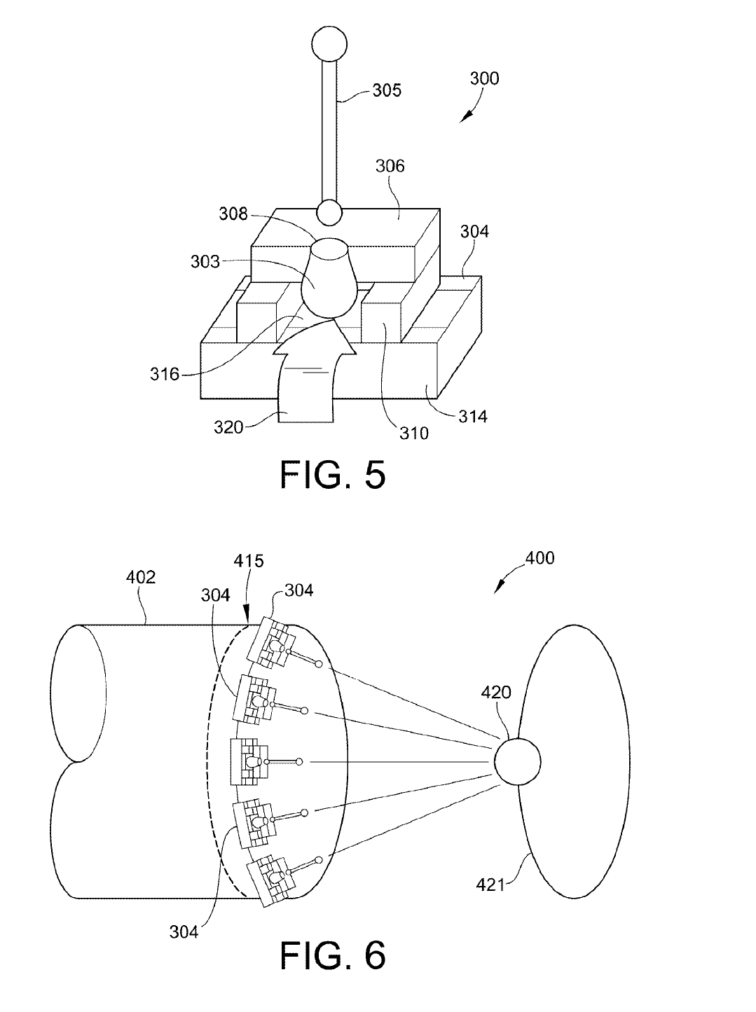

FIG. 6 shows an embodiment of a medical device 400 having a medical tool portion 402, in the form of a surgical knife, and an ablative device portion 415 with a plurality of firing chambers 304 configured to fire ablative fluid in different directions according to this disclosure. Each firing chamber 304 is configured to propel a fluid from inside the firing chamber through an orifice and toward target material 420, or area to be treated 421, to ablate target material 420 according to a programmed pattern of ablative treatment for target material 420 or treatment area 421. The manner of operation of each firing chamber to ablate the target has been previously described.

Medical device 400 may have a plurality of orifices, each one or more of the plurality of orifices being associated with a firing chamber 304. As shown in FIG. 6, each of the plurality of firing chambers is individually aligned to have its orifice and hence axis of fluid propulsion directed toward the target material 420. Hence, the axis of propulsion of each firing chamber is at an angle to the plane of the ablative device in this example. The angle of each axis may be fixed for the ablative device. Alternatively, the angle of each axis may be configured to be adjustable using robotic mechanisms well known in the art. For example sensors and drive mechanisms may be provided to one or more firing chambers for the purpose of adjusting one or more firing chambers with respect to a target. A controller may detect the angle of the axis of propulsion with respect to the plane of the ablative device portion and with respect to the coordinates of the target and provide instructions to the drive mechanisms to adjust the angle of the one or more firing chambers with respect to the target. In at least one embodiment, ablative device portion 415 is flexible, and medical tool portion 402 is configured to change the shape of ablative device portion in response to a change in pressure inside medical tool portion 402. These embodiments may provide the ablative device of this disclosure with more variable control for providing more precise ablative procedures.

FIG. 7 shows medical device 500 having an ablative device portion 516 which is incorporated into a medical tool portion 502 and used on a patient 514 in a medical procedure.

As seen in the exploded view shown in FIG. 7, ablative device portion 516 includes a plurality of independently controllable firing chambers 504. In the further exploded view shown in FIG. 7, each independently controllable firing chamber 504a is in fluid communication with fluid from a source of fluid through a fluid line 507. Each independently controllable firing chamber 504a is configured to propel fluid from inside the firing chamber toward a target material to ablate the target material. Medical device 500 may comprise or be associated with a controller (not shown) configured to independently control each of the independently controllable firing chambers 504 according to a programmed pattern as previously described in FIGS. 4A-4D. The controller may be associated with a memory unit, wherein the programmed pattern is an instance of instructions stored in the memory unit, and wherein the control of firing chambers 504 occurs by the controller executing the instructions. In this illustrative example, the controller may be located inside the ablative device portion 516 as described in FIGS. 3A, 3B above. Alternatively, the controller may be the controller explained in FIG. 2 above. In either case, signal lines 505 and 506 provide signal paths for controlling the controller.

As shown in FIG. 7, medical device 500 is being used as a steerable catheter. As used herein, a “catheter” is a medical device that is inserted into a cavity of the body typically to withdraw or introduce fluid. The catheter typically includes a shaft which may contain one or more lumens. The shaft may be bendable and/or steerable. The catheter may be inserted into a subject for introduction of fluids, for removal of fluids, or both. The subject may be a vertebrate subject such as a mammalian subject. Catheters may be soft catheters which are thin and flexible or may be provided in varying levels of stiffness depending on the application. Catheters may be inserted in the body to treat diseases or perform a surgical procedure. By modifying the material or adjusting the way catheters are manufactured, catheters may be tailored for a wide range of medical uses including cardiovascular, urological, gastrointestinal, neurovascular, ophthalmic, and other medical applications. Some commonly used catheters include peripheral venous catheters, which may be inserted into a peripheral vein, usually in the hand or arm, for the administration of drugs, fluids, and so on.

As used herein, a catheter may include various accessory components, subassemblies, or other accessory parts. For instance, a catheter may include molded components, over-molded components, subassemblies, or other accessory components or parts. The catheter may also include connecting fittings such as hubs, extension tubes, and so on. Various catheter tips designs are known. These designs include stepped tips, tapered tips, over-molded tips and split tips for multi-lumen catheters, and so on. Other components or accessories that may be associated with a catheter may include one or more lights, cameras, or other components that may aid in steering the ablative device of the present disclosure toward a target for ablating. In at least one embodiment, a catheter has a flexible, bendable, or steerable shaft.

The medical device 500 may be a catheter wherein medical device portion 502 is in the form of a steerable cylinder and ablative device portion 515 is in the form of a ring 516 that is attached to an end of the medical device portion 502. Alternatively, ablative device portion 515 may be integrated with the medical portion 502 into a medical device 500 such that the one or more firing chambers 504 are integrated into the medical device.

In the configuration shown in FIG. 7, medical device 500 further comprises a removal channel 503 configured to remove ablated material and fluid from a subject. More particularly, fluid introduced into a subject with firing chambers 504 may be immediately removed through removal channel 503. Illustratively, a vacuum placed upon removal channel 503 by a vacuum device (not shown) may be used to create a pressure differential with the site of the target for drawing the ablated material and fluid through channel 503 into the vacuum device for disposal.

In at least one embodiment of the present disclosure, the medical device is configured to immediately remove ablated material produced in the body. For example, material such as cancerous material, “foreign” material, or other material which may include cells that if left inside the body may metastasize, may be immediately removed from the body. This is especially important if the ablated material contains cancer cells since cancer cells may be detrimental to the body. Dislodged cancer cells not removed may allow those cancer cells to migrate elsewhere in the body where they may metastasize and cause harm to the patient. If the ablated material is biologically friendly and absorbable by the body, removal may still be desirable but may not always be required.

The previously described removal channel 503 formed by the medical tool portion of the medical device shown in FIG. 7 provides one structure and method for the removal of ablated material from the body. There are others. For instance, in an alternative illustrative embodiment, the ablative tool portion of the medical device may be used with systems that may remove the ablated material by suction, drainage, vacuum removal, aspiration, and/or other techniques.

FIG. 7 also shows that medical device 500 may also include a sensor 511. Sensor 511 may be configured to sense at least one parameter of the target material being removed, or having been removed and accumulated, through removal channel 503. The controller may be in communication with sensor 511 and the controller may be configured to control the ablative device portion 516 in response to the sensed parameter. Sensor 511 may be configured to sense at least one parameter associated with an ablative treatment for controlling the ablative device portion 516. For example, sensor 511 may be configured to sense at least one parameter such as temperature of propelled fluid, pressure of propelled fluid, temperature of a cavity at various points in time or throughout the ablative treatment, pressure in a cavity at various points in time or throughout the ablative treatment, temperature of target material removed by the ablative device portion, and amount of target material removed by the ablative device portion. Additionally, medical device 500 may be configured with a camera and a light source for viewing the target material removed from the body and/or the target material during a medical procedure. The camera may be configured with a lens that is positioned along the plane of the ablative device portion to allow a caregiver to view the ablative procedure using the ablative device of this disclosure.

In at least one illustrative embodiment of medical device 500, ablative device portion 516 comprises microelectromechanical systems (MEMS) and has nano-scale technologies for biomedical applications. For example, medical device 500 may comprise a surgical tool configured for surgical procedures such as neonatal, coronary, ophthalmic, gallstone treatment, liver resection, or plastic/cosmetic procedures. Medical device 500 may comprise a catheter and may be configured for performing minimally invasive surgery. For example, medical device 500 may have one or more actuators, sensors, or imaging devices and may provide a cutting tool for micro-biopsies.

In at least one additional illustrative embodiment, medical device 500 comprises biomedical microsystems for minimally invasive treatment and optionally diagnosis. For example, medical device 500 may comprise a catheter and may be configured for entering a blood vessel of a patient and steered toward a target material in the heart. Ablative device portion 516 may be a disposable MEMS-based micro-biopsy catheter configured for minimally invasive tissue sampling or ablating.

Medical device 500 may be configured for steering of an ablative treatment in several ways. For example, ablative device portion 516 may be flexible and may be configured to change shape during a medical procedure. For example, orifices may be configured to have a variable axis of propulsion as disclosed with reference to FIG. 6. Steering may also be accomplished by varying the addressing or activating one or more patterns of firing chambers, as disclosed with reference to FIGS. 4A-4D. Additionally, steering may be accomplished by bending or flexing medical tool portion 502. As previously explained, steering may also be accomplished using robotics and in other ways that will be apparent to one skilled in the art in view of this disclosure.

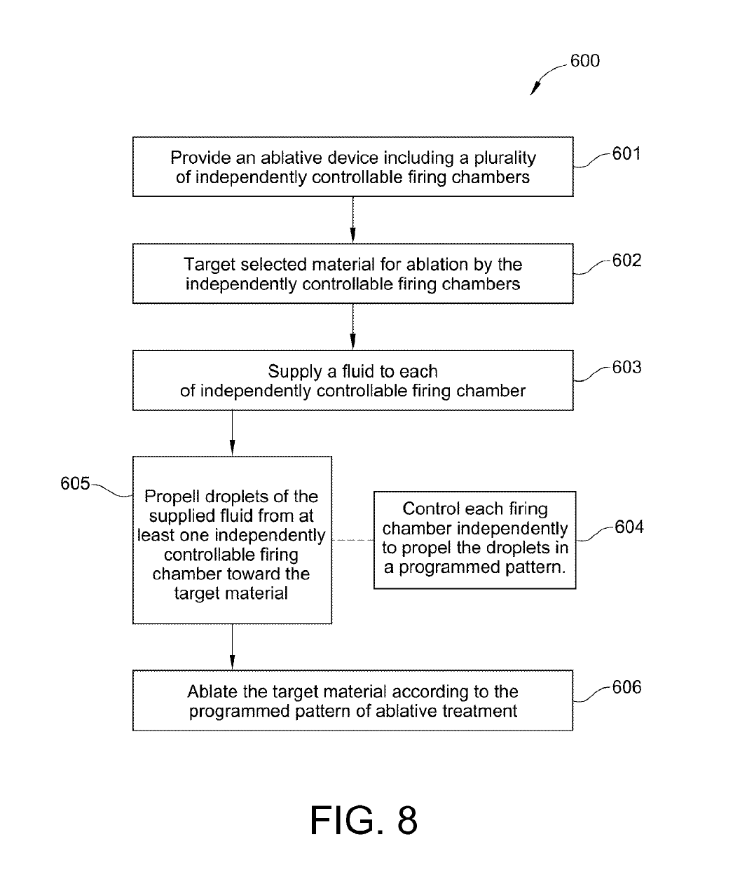

FIG. 8 is a flowchart illustrating method 600 of ablating a targeted material with an ablative device according to the present disclosure. Method for ablating a material 600 comprises providing an ablative device including a plurality of independently controllable firing chambers at step 601. A selected material for ablation is targeted with the independently controllable firing chambers at step 602. Each independently controllable firing chamber is supplied with a fluid at step 603. Steps 604 and 605 may be carried out substantially simultaneously wherein each firing chamber is controlled and at least some of the firing chambers are controlled to propel droplets of the supplied fluid toward the targeted material. The method may end with ablating the target material according to the programmed pattern of ablative treatment at step 606. The propelling of droplets of the supplied fluid from at least one independently controllable firing chamber may be carried out by superheating a thin layer of the fluid in the at least one firing chamber.

Other and additional steps may be carried out in method 600. For example, a pattern of propelling droplets from firing chambers at step 605 may be selected and may be changed while ablating a target material. The controlling of each firing chamber at Step 604 may be carried out with a controller and the programmed pattern may be changed while ablating. Additionally, a sensor may also be provided at step 601. The sensor may sense one or more parameters associated with ablating, such as temperature of propelled fluid, pressure of propelled fluid, temperature of a cavity in the target material formed by the action of the propelled fluid, pressure in a cavity at various points in time or throughout the ablative treatment, temperature in a cavity at various points in time or throughout the ablative treatment, and amount of target material removed by the ablative device portion. A controller may then control, at step 604, each firing chamber in response to a sensed parameter.

In view of this disclosure, it will be seen that technologies are generally described for a method and a device for ablating a targeted material. Disclosed herein is an ablative device having a plurality of independently controlled firing chambers configured to ablate a targeted material.

Other and alternative aspects of the present disclosure may provide an ablative device configured to ablate a target material. For example, various aspects of the present disclosure may provide a method of ablation that could be applied in a miniaturized and localized manner and that doesn't result in substantial collateral damage to nearby healthy tissues. Aspects of the present disclosure may assist a physician in balancing the competing requirements between inadequate break-up or removal of the unwanted material and unnecessary removal of healthy surrounding tissue, as either over-treatment or under-treatment may result in undesirable patient outcomes. Aspects of the present disclosure may provide for improved techniques that may allow surgeons to treat gallstones, cancerous tumors, and related conditions with improved outcomes. Such improvements may result from using the presently disclosed device and method of ablation that may be applied in a miniaturized and localized manner.

In at least one aspect of the present disclosure, an ablative device is configured using inkjet printing technology. Inkjet printing is a type of computer printing that creates a digital image by propelling droplets of ink onto paper. Inkjet printers are a commonly used type of printer. For example, an inkjet printing device may be used as an ablative device and water or other ablating fluid, instead of ink for printing, may be in fluid communication with the inkjet printing device.

Illustrative technologies for use in configuring the ablative device of this disclosure includes the two main technologies in use in contemporary inkjet printers, continuous (CIJ) and Drop-on-demand (DOD). Drop-on-demand (DOD) is divided into thermal DOD and piezoelectric DOD. In the thermal inkjet process, the print cartridges contain a series of tiny chambers, each containing a heater, all of which are constructed by techniques known to those skilled in the art, including photolithography. To eject a droplet from each chamber, a pulse of current is passed through the heating element causing a rapid vaporization of the ink in the chamber to form a bubble, which causes a large pressure increase, propelling a droplet of ink onto the paper. The ink's surface tension, as well as the condensation and thus contraction of the vapor bubble, pulls a further charge of ink into the chamber through a narrow channel attached to an ink reservoir. The inks used may have a volatile component to form the vapor bubble, otherwise droplet ejection may not occur. In piezoelectric DOD, a piezoelectric material in an ink-filled chamber is behind each nozzle, instead of a heating element. When a voltage is applied, the piezoelectric material changes shape, which generates a pressure pulse in the fluid forcing a droplet of ink from the nozzle. Piezoelectric inkjet may allow for a wider variety of inks or fluids than thermal inkjet as there is no need for a volatile component, and little or no issue with kogation (buildup of ink residue). A DOD process uses software that directs the heads to apply between zero to eight droplets of ink per dot, only where needed.

The continuous inkjet (CIJ) technology comprises a high-pressure pump that directs liquid ink from a reservoir through a gunbody and a microscopic nozzle, creating a continuous stream of ink droplets via the Plateau-Rayleigh instability. A piezoelectric crystal creates an acoustic wave as it vibrates within the gunbody and causes the stream of liquid to break into droplets at regular intervals: 64,000 to 165,000 droplets per second may be achieved. The ink droplets are subjected to an electrostatic field created by a charging electrode as they form; the field varies according to the degree of drop deflection desired. This results in a controlled, variable electrostatic charge on each droplet. Charged droplets are separated by one or more uncharged “guard droplets” to minimize electrostatic repulsion between neighboring droplets.

The charged droplets pass through an electrostatic field and are directed (deflected) by electrostatic deflection plates to print on the receptor material (substrate), or allowed to continue on undeflected to a collection gutter for re-use. The more highly charged droplets are deflected to a greater degree. Only a small fraction of the droplets is used to print, the majority being recycled.

In at least one other aspect of the present disclosure, ablative techniques for use in gallstone, cancer, cardiac, liver resection, colonoscopy, cauterization, cardiac, gastrointestinal (GI) tract and pulmonary surgery are disclosed. In at least one additional aspect of the present disclosure, an ablative device and technique to improve treatment of gallstones and tumors through localized ablation using miniaturized devices similar to advanced MEMS thermal inkjet printheads is provided. The devices are used to superheat a very thin layer of fluid in microseconds. The sudden vaporization propels droplets of liquid at high speed to ablate cells in a high resolution, well-controlled and targeted hydromechanical type of treatment. Because the ablation is performed by the impingement of the droplets (like water blasting), tissue damage from overheating may be avoided. Because the targeting is precise, the risks of under-treatment and over-treatment inherent in previous techniques, as shown in FIG. 1, may be greatly reduced.

The ablative device may be built into a localized surgical tool, and furthermore, ablative treatment may also be coupled with aspiration or a stent to facilitate drainage and removal of the material that has been broken up with an inkjet-like ablative technique. A stent is a mold or a device of suitable material used to provide support for structures for holding one or more biomaterials or biostructures in place. These biomaterials and biostructures may include skin, arteries, bodily orifice or cavity, or other biomaterial or biostructure of the body of the subject into which the stent may be placed. Illustrative stents may include biliary, urethral, ureteral, tracheal, coronary, gastrointestinal, esophageal stents, and so on. Stents may be used to treat coronary artery disease, problems in the peripheral vascular system, bile ducts and biliary tree, kidney, urinary tract, trachea, and bronchi. Stents may also be used to treat other medical conditions. The stents may be of any shape or configuration. The stents may include a hollow tubular structure, which may be useful in providing flow or drainage through ureteral, biliary, or other lumens. Stents may be coiled or patterned as a braided or woven open network of fibers, filaments, and so on. Stents may also include an interconnecting open network of articulable or other segments. Stents may have a continuous wall structure or a discontinuous open network wall structure.

As used herein, a stent may include a stent cover which may include a tubular or sheath-like structure adapted to be placed over a stent. The stent cover may include an open mesh of knitted, woven or braided design. The stent may be made of any material useful for providing structure for holding one or more biomaterials or biostructures in place. These materials may include metallic and non-metallic materials. They may also include shape memory materials. Metallic materials may include shape memory alloys such as nickel-titanium alloys. They may also include other metallic materials such as stainless steel, tantalum, nickel-chrome, cobalt-chromium, and so on.

While in the illustrative example, the propulsion of fluid is effected by use of a controllable resistive heating elements, it will be appreciated that there are other ways known to one skilled in the art for controlling the propulsion of the fluid from independently controllable firing chambers of this disclosure. For example, each one of the independently controllable firing chambers may be provided with a controlled valve provided in-line with an orifice of the orifice plate that has been associated with that independently controllable firing chamber. The valve may be, illustratively, normally closed to keep fluid inside that independently controllable firing chamber

Under the influence of an electric signal in accordance to a predetermined signal pattern, the controlled valve may be opened up to allow for the discharge of fluid inside that independently controllable firing chamber out through the orifice toward the target to be ablated. In this example, fluid is provided to that independently controllable firing chamber at a high enough pressure that on opening of the controlled valve, fluid may be delivered to the site of the target material at forces sufficient for the intended ablative treatment.

In an alternative embodiment, the foregoing high pressurized fluid valve system may be used in combination with a thermal activator such as the resistive heating elements previously described to deliver the intended ablative treatment.

In another illustrative example, the propulsion of fluid is effected by a piezoelectric material provided in each of the independently controllable firing chambers. The piezoelectric material is positioned inside each one of the independently controllable firing chambers. In this example, when a voltage is applied, the piezoelectric material changes shape, which generates a pressure pulse in the fluid inside each independently controllable firing chamber forcing a droplet of fluid through a one orifice of the array of orifices. In addition, each piezoelectric material may create an acoustic wave as it vibrates within the independently controllable firing chamber in which it is placed and cause a stream of liquid to break into droplets at regular intervals. For example, 64,000 to 165,000 droplets per second may be propelled from an independently controllable firing chambers provided with a piezoelectric material for use in actuating the propulsion of fluid toward an ablative target.

An activator comprising piezoelectric material may allow for a wider variety of fluids to be used with this disclosure than a thermal activator comprising the resistive heating elements, described above and may decrease any tendency of residue accumulation in each independently controllable firing chamber and associated orifices that may occur with other activators. Other electro-mechanical activators and combinations of activators may be used with this disclosure to allow for the patterned discharge of fluid from the ablative device of this disclosure.

Aspects of the ablative device and method of the present disclosure may allow for treatment of gallstones, tumors and related conditions through a localized MEMS thermal inkjet-like ablation technique using miniaturized devices that may be brought to the area to be treated as part of a localized surgical tool. This may be used in open surgery, or as part of a catheter tip or other endoscopic or laparoscopic device.

MEMS thermal inkjet (TIJ) printing technologies may be used in modeling how a liquid droplet may be propelled by the use of superheat on a micro-scale in an extremely well-targeted and high resolution way in the disclosed ablative device. The energy for the localized ablation arises from the very controllable superheat explosion of the picoliter-scale volume of fluid (2 pL is typical in modern TIJ printheads) in the thermal inkjet-like device, which may not damage surrounding tissues by overheating, overcooling, overgrinding, etc. In a localized area within the individual firing chambers, the liquid in a very thin layer is heated to approximately 300° C. over microseconds, within an extremely small volume of the fluid immediately adjacent to the resistive heater in the firing chamber, producing an energetic explosion controlled and directed over an extremely small area.

The area to be ablated may be controlled from sizes analogous to a few pixels of printing to the sizes of hundreds, thousands, or more pixels activated in parallel to provide ablation over a larger controlled area. An exemplary instance of this invention can be described using the specifications of a commercially available inkjet printhead which has a 1200 dpi resolution, equivalent to 21 μm per dot. If one chamber were fired, it could be used to target an area corresponding roughly to a 20 μm diameter dot, and if 100 contiguous chambers were fired, an area roughly 100 times larger could be targeted. If all the chambers of this device were fired at once, it would cover an area roughly equivalent to the total printhead active area of 0.5″×0.85″, which is roughly 1.3 cm×2.2 cm. This area may be large enough, and at the same time may have enough resolution to be suitable for surgical applications.

The device of the present disclosure may have similarities to inkjet printing, in that it may be conducted at conditions that superheat a very thin layer of the liquid before vaporization, which adds to the energy of the vaporization and the reproducibility of the rapid vaporization. This is achieved by very rapid localized heating, which also reduces the power needed overall for vaporization as lateral heat transfer losses are minimized over the rapid time scale used.

In at least one aspect of the ablative device of the present disclosure, the use of a small resistive heater made in a thin film technology analogous to inkjet technology, for which 0.2 W of extractable energy may be obtained from a 37 V pulse applied over 6 microseconds. In the current disclosed ablative device, such energy may be used to propel droplets of liquid at the treatment site to generate an extremely localized energetic hydromechanical ablation technique.

Aspects of the presently disclosed ablative device may be employed at a distance from the area to be treated, which may render the tool of far greater utility than if contact were required, allowing the ability for the surgeon to observe what the treatment site looks like during the treatment session. Extractable energy from a firing chamber of the present disclosure may generate a significant acoustic pressure wave of 200 mBar (0.2 Atm) at its peak, at 2 mm away from the printhead. In at least one other aspect, in which a longer Pt wire is used to apply the superheat to the liquid thin layer surrounding the wire, an electrical energy input of 24 W for 8 microseconds may yield 0.5 W of extractable energy, and 6.2 Bar (6 Atm) of pressure. An acoustic pressure wave of 25 mBar may be produced at its peak at 20 mm (2 cm) away. Such pressure waves may be achieved even without including any particular device structure designed to intentionally direct the pressure wave or propelled liquid. An aspect of the medical device of the present disclosure may be designed with greater directionality to allow operation with up to cm-scale distances separating the hydromechanical inkjet-like device and the stone or cancerous tissue to be treated.

In at least one additional aspect of the present disclosure, a resistive heater in firing a chamber rapidly heats a liquid (which could be water, water with additives, or other liquids) over a time period of roughly microseconds so as to provoke homogeneous nucleation (superheating) before any significant lower-energy heterogeneous nucleation (traditional boiling as seen in cooking) has a chance to occur. Localized pressures of about 130 atmospheres may be generated. For example, an ablative device of the present disclosure may have 36,000 nozzles or orifices and be configured to operate at about 48 kHz to provide 1200 dots per inch (dpi) with 2 pL droplets, in roughly a 0.5″×0.85″ area.

Aspects of the presently disclosed medical device use the highly energetic and rapid, even explosive, vaporization enabled by the rapid rate of heating of a resistor, and may be used to direct the energy to the gallstone or tumor area under treatment. The extremely small pixels used in inkjet printing technology may allow for extremely localized heating and explosion, and the ability for localized steering and individual addressing of a section of nozzles to treat the exact area desired may make a presently disclosed TIT-like surgical tool especially useful to target treatment of affected diseased areas with minimal peripheral damage to healthy tissues. These ablative techniques may be particularly of use for very localized tumor ablation, where surrounding healthy tissue must not be damaged, such as for treatment of liver tumors.

In at least one aspect of the presently disclosed medical device, a MEMS thermal inkjet-like device, which may be catheter-based with a desired number of inkjet like firing chambers, may be used with its own micro-reservoir of clean liquid in order to reduce any fouling of the chambers or thermal resistors. It may furthermore be desirable to provide a medical device using relatively soft materials, such as flexible organic electronics rather than silicon, wherever possible, both to minimize any scraping damage in tight treatment locales, and to maximize the ability to wrap the treatment device around portions of a treatment catheter-type tool.

In at least one aspect of an ablative device of the present disclosure, having a flexible substrate or finely divided areas of a rigid substrate such as silicon, a focal effect may be incorporated into the device where an array of one to several to hundreds of highly directed ‘pixels’ of inkjet-like devices can be focused onto a target material such as a stone or tumor area, to allow for added selectivity and power in treatment. For example, an ablative device of the present disclosure may be adapted with a catheter or surgical ‘knife’ to treat tissue areas ranging from sub-cm to cm and larger by selecting an array size and addressing of the inkjet-like firing chambers used, as needed in the treatment.

Besides tissue ablation, another application of the present disclosure may be the prevention or removal of potential obstructions in stents. In this mode, a stent may be placed to provide an alternate route for drainage from the gall bladder or other affected areas, but painful gallstone obstructions may find their way in to occlude the stent over time. In this aspect, a localized MEMS inkjet-like ablative device may be placed in or near the stent to allow localized break-up and/or ablation of the blockage or blockage precursor to enhance its removal, and to keep the stent clear while serving its function of enhancing drainage.

In at least one aspect of the present disclosure, an ablative device is configured for the use biologically active ablation liquids, designed for specific purposes. For example, if the surgeon is ablating an infected mass, it may be advantageous to use a bactericidal liquid such as pure ethanol alcohol. If the surgeon is ablating a cancerous mass, it may be advantageous to employ a specific chemical used in chemotherapy cancer treatment. If the surgeon is ablating an inflamed heart or intestinal tissue, or other inflamed tissue, it may be advantageous to use an anti-inflammatory liquid drug. Additionally, the use of an anesthetic liquid may be useful to reduce the need for systemic use of anesthetic drugs. To prevent fouling, the orifice may be flushed with pure water after use.

Aspects of the present disclosure may have an aspiration or removal channel which may furthermore be incorporated into a treatment device such as a catheter. One or more sensors may be provided with the medical device of the present disclosure. Sensor(s) may be configured for immediate, or nearly immediate, diagnostic feedback to determine the margins of the diseased tissues. For example, onboard sensors or remote analysis of aspirated materials with nearby rapid laboratory diagnostic evaluations may provide real time feedback to a surgeon performing surgery with the presently disclosed ablative device.

Aspects of the present disclosure may be applicable to gallstones, kidney stones, and other painful obstructions in the body. In addition, the ablation techniques and devices disclosed herein may be useful as a more localized and less damaging alternative to more traditional ablation techniques currently used on tumors, such as in liver resection as shown in FIG. 1. The presently disclosed ablative device and technique may be particularly well-suited to treating conditions such as gallstones, and in cancer treatments to aid in liver resection, colonoscopy, cauterization, and cardiac, GI tract and pulmonary surgery.

Ablative techniques disclosed herein may be of use in gallstone, cancer, and cardiac treatments often as a part of minimally invasive surgical techniques, with the goal of treating these diseases with less overall trauma to the patient and lower cost. Ablative techniques including powerful lasers, rotary gears and saws are some of the standard practices for reduction and removal of gallstones and cancerous tumors. These techniques may be imprecise, and can cause considerable collateral damage to the patient. A more localized technique, with less potential for damage, may be presently provided by this disclosure.

Ablative technologies disclosed herein may be thermal or non-thermal in nature. Aspects of the present disclosure may lie somewhat between these techniques in that it may use superheat of a very thin layer of fluid (thermal) to propel droplets of liquid in an extremely targeted way to ablate cells in a way that may be thought of as a hydromechanical (non-thermal) treatment. Aspects of the presently disclosed medical device may incorporate technology similar to MEMS thermal inkjet printing technology. This may provide for the propulsion of droplets by the micro-scale vaporization of a small portion of the fluid, to result in energetic movement in an extremely well-targeted and high resolution way.

Aspects of the present disclosure provide an ablative device made with MEMS thermal inkjet-like (TIJ) devices, such as TIJ firing chambers, and may thus be compact and amenable to mass production. This may lower the cost and the device may be small and easily used to target very specific areas with minimal collateral damage, which may improve its utility.

The presently disclosed technique may be of great use in walking the fine line that challenges many surgical and ablative techniques, between inadequate break-up or removal of the unwanted material on one hand, and unnecessary removal of healthy surrounding tissue on the other. Either over-treatment or under-treatment can result in undesirable patient outcomes, and the precision and scalability of this invention may enable just the right amount of tissue removal, resulting in more beneficial surgeries, and improved patient outcomes.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those with skill in the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-43. (canceled)

44. A medical device for use in performing a medical procedure comprising:
a medical tool portion;
an ablative device portion connected to the medical tool portion;
a source of fluid in fluid communication with the ablative device portion; and
the ablative device portion including a plurality of independently controllable firing chambers, wherein:
each of the firing chambers is in fluid communication with the source of fluid; and
each of the firing chambers comprises a heater element of a plurality of heater elements, the heater element configured to heat a portion of the fluid within the firing chamber to propel some of the fluid within the firing chamber toward a target material to ablate the target material according to a programmed pattern of ablative treatment for the target material.

45. The medical device of claim 44 further comprising a controller configured to independently control the propulsion of some of the fluid from within each of the firing chambers toward the target material according to the programmed pattern.

46. The medical device of claim 45 further comprising a memory unit, wherein the programmed pattern is an instance of instructions stored in the memory unit, and wherein the control of the propulsion of some of the fluid from within each of the firing chambers toward the target material occurs by the controller executing the instructions.

47. The medical device of claim 45 wherein:
the ablative device portion further comprises a resistive heater array including the plurality of heater elements in which each of the heater elements comprises a resistive heater element;
each resistive heater element is configured to superheat a thin layer of the fluid within one of the firing chambers under the influence of an applied electric current; and
the control of the propulsion of some of the fluid from within each of the firing chambers toward the target material occurs by selective heating of one or more of the plurality of the resistive elements in response to electrical signals applied by the controller according to the programmed pattern.

48. The medical device of claim 45 wherein the ablative device portion further comprises a removal channel configured to provide a removal path for the fluid following propulsion of some of the fluid from within at least one of the plurality of firing chambers of the ablative device portion and at least a portion of the target material that is ablated.

49. The medical device of claim 48 further comprising a sensor configured to sense at least one parameter of material removed through the removal channel.

50. The medical device of claim 49 wherein the controller is configured to control at least one operating factor of the ablative device portion in response to the sensed at least one parameter.

51. The medical device of claim 44 wherein the ablative device portion comprises:
a substrate configured to attach to the medical tool portion and defining bottom walls of the plurality of firing chambers;
a heater array on or in the substrate comprising the plurality of heater elements for generating predetermined heating patterns under the influence of predetermined electric current patterns applied to the plurality of heater elements according to the programmed pattern;
a barrier grid on the substrate defining sidewalls of the plurality of firing chambers; and
an orifice plate on the barrier grid defining top walls of the plurality of firing chambers, the orifice plate comprising an array of orifices, each orifice of the array of orifices being associated with one of the plurality of the firing chambers, each orifice being configured to direct the propulsion of some of the fluid from inside the associated firing chamber toward the target material according to the programmed pattern.

52. The medical device of claim 51 wherein the substrate is formed from a flexible material.

53. The medical device of claim 51 wherein the substrate includes flexible electronics.

54. The medical device of claim 51 wherein the array of orifices includes at least two orifices, and wherein a first of the at least two orifices is configured to point in a first direction and a second of the at least two orifices is configured to point in a second direction that intersects with the first direction in order to directionally control the propelling of some of the fluid from within at least two of the firing chambers toward the target material.

55. The medical device of claim 51 further comprising:
a second source of fluid in fluid communication with the ablative device portion of the medical device; and
wherein at least one firing chamber is in fluid communication with the second source of fluid.

56. The medical device of claim 51 further comprising a signal path to each heater element for communicating an electrical signal to each heater element.

57. The medical device of claim 56 further comprising a controller for applying the electrical signal to each heater element according to the programmed pattern.

58. The medical device of claim 57 wherein the substrate comprises the controller.

59. The medical device of claim 44 wherein each heater element is configured to superheat a thin layer of the fluid within one of the firing chambers to thereby propel droplets of the fluid from the firing chamber at a speed and pressure sufficient to ablate cells.

60. The medical device of claim 44 wherein each heating element is configured to rapidly heat a thin layer of the fluid within one of the firing chambers to a temperature of about 300° C.

61. The medical device of claim 44 wherein the medical tool portion comprises a catheter tip, endoscopic device, laparoscopic device, or stent.

62. A method for ablating a material comprising:
providing an ablative device including a plurality of independently controllable firing chambers;
supplying a fluid to each independently controllable firing chamber of the plurality of independently controllable firing chambers;
independently controlling each firing chamber according to a programmed pattern of ablative treatment for a target material;
superheating a thin layer of the fluid in at least one firing chamber to propel some of the fluid from inside the at least one firing chamber toward the targeted material; and
ablating the target material according to the programmed pattern of ablative treatment.

63. The method for ablating a material of claim 62 wherein the target material is organic tissue in a body.

64. The method for ablating a material of claim 63 further comprising a step of removing ablated organic tissue from the body, upon performing the step of ablating the organic tissue.
